(19) United States
(12) Patent Application Publication
(10) Pub. No.: US 2008/0241831 A1
(43) Pub. Date: Oct. 2, 2008

(54) Methods for detecting small RNA species

(76) Inventors: Jian-Bing Fan, San Diego, CA (US); Jing Chen, San Diego, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/729,755

(22) Filed: Mar. 28, 2007

(51) Int. Cl. C12Q 1/68

(52) U.S. Cl. 435/6

(57) ABSTRACT

The invention provides a method of detecting small target nucleotide sequences, in particular, small RNA species that are present in a sample. The method generally comprises a poly-A polymerization step or a ligation step to add a universal sequence to the 3′-end of all RNA molecules, followed by a universal primer-mediated cDNA synthesis, solid-phase selection, assay oligo annealing, extension and PCR amplification/labeling. The method of the invention can be practiced to amplify and label a small amount of miRNA or other ncRNA. The resulting amplification product can be read out on a universal array or an array with miRNA-specific or ncRNA-specific probes. The invention has multiple embodiments, including methods, compositions, and kits. In general, the nucleic acids, compositions, and kits comprise materials that are useful in carrying out the methods of the invention or are produced by the methods, and that can be used to detect small target nucleic acid sequences present in samples, in particular, small RNA species.

### BACKGROUND OF THE INVENTION

The present invention relates to improved detection methods for small target nucleic acid sequence targets, including micro RNA (miRNA), small interfering RNA (siRNA) and other small non-coding RNAs (ncRNAs).

There has been great interest in the analysis of small RNAs, such as short interfering RNAs (siRNAs), microRNAs (miRNA), tiny non-codingRNAs (tncRNA) and small modulatory RNA (smRNA), since the discovery of siRNA biological activity over a decade ago. Traditionally, most RNA molecules were thought to function as mediators carrying the information from the gene to the translational machinery. The most prominent exceptions to this, transfer RNA (tRNA) and ribosomal RNA (rRNA), both of which are involved in the process of translation. However, since the late 1990s, it has been widely acknowledged that other types of untranslated RNA molecules are present in many different organisms ranging from bacteria to mammals, and are affecting a large variety of processes including plasmid replication, phage development, chromosome structure, DNA transcription, RNA processing and modification, development control and more. These untranslated RNA molecules have been given a variety of names, the term small RNAs (sRNAs) predominantly used for bacterial RNAs while the term noncoding RNAs (ncRNAs) has been more common in eukaryotes.

The term non-coding RNA (ncRNA) is commonly employed for RNA that does not encode a protein, but this does not mean that such RNAs do not contain information nor have function. Although it has been generally assumed that most genetic information is transacted by proteins, recent evidence suggests that the majority of the genomes of mammals and other complex organisms is in fact transcribed into ncRNAs, many of which are alternatively spliced and/or processed into smaller products. These ncRNAs, such as microRNAs and siRNAs, regulate gene expression at multiple levels including chromatin architecture, transcription, RNA editing, RNA stability, and translation. ncRNAs, including those derived from introns, appear to comprise a hidden layer of internal signals that control various levels of gene expression in physiology and development, including chromatin architecture/epigenetic memory, transcription, RNA splicing, editing, translation and turnover. RNA regulatory networks may determine most of our complex characteristics, play a significant role in disease and constitute an important source of genetic variation both within and between species.

miRNAs are transcribed as precursors (pri-miRNAs) that are processed in the nucleus and cytoplasm to generate RNP complexes containing 21-nt miRNAs that are partially complementary to the 3′ untranslated region (UTR) of mRNAs. Binding of RISC-miRNA complexes inhibits translation of the cognate mRNA, thus silencing gene expression. Despite the challenge of finding bona fide miRNAs and miRNA targets based on limited sequence complementarity, computational and tailored cloning efforts are providing a growing list of miRNAs in multicellular organisms. Frequently, one miRNA can target multiple mRNAs and one mRNA can be regulated by multiple miRNAs targeting different regions of the 3′ UTR. Conversely, miRNA binding sequences are absent from the 3′ UTR of genes involved in basic cellular processes or of genes coexpressed with particular miRNAs. These features allow coordinated regulation, combinatorial control and precision and robustness to an increasing number of cell fate decisions and developmental transitions.

miRNAs can therefore act as regulators of cellular development, differentiation, proliferation and apoptosis. miRNAs can modulate gene expression by either impeding mRNA translation, degrading complementary mRNAs, or targeting genomic DNA for methylation. For example, miRNAs can modulate translation of mRNA transcripts by binding to and thereby making such transcripts susceptible to nucleases that recognize and cleave double stranded RNAs. miRNAs have also been implicated as developmental regulators in mammals in two recent mouse studies characterizing specific miRNAs involved in stem cell differentiation. Numerous studies have demonstrated miRNAs are critical for cell fate commitment and cell proliferation. Other studies have analyzed the role of miRNAs in cancer. miRNAs may play a role in diabetes and neurodegeneration associated with Fragile X syndrome, spinal muscular atrophy, and early on-set Parkinson's disease. Several miRNAs are virally encoded and expressed in infected cells.

Recent reports have revealed important roles of miRNAs in the development of human cancers. The levels of about 200 miRNAs correlated with lineage and differentiation of tumor cells, and were significantly better criteria to classify poorly differentiated tumors than expression profiling of more than 2000 protein-coding genes, arguing for pivotal roles of miRNA levels in tumor development. Clusters of miRNAs have the properties of classical oncogenes, and modulate—and are modulated by—the activities of other oncogenes. For example, a regulatory network was recently discovered in which increased transcription of a cluster of miRNAs by the proto-oncogene c-MYC results in translational down regulation of the transcription factor E2F1, another important regulator of cell division, which is itself transcriptionally regulated by c-MYC. Thus, miRNAs could serve in this case as part of a safety mechanism that adjusts the levels of expression of a key regulator of cell cycle progression. Analysis of the role of miRNA in these processes, as well as other applications, would be aided by the ability to more accurately and specifically detect and measure miRNA. However, the small size of the miRNAs makes them difficult to quantify using conventional prior art methods.

There exists a need for highly specific and sufficiently sensitive methods and systems for detecting and quantitating miRNA. The present invention satisfies this need and provides related advantages as well.

### SUMMARY OF THE INVENTION

The invention provides a method of detecting small target nucleotide sequences, in particular, small RNA species that are present in a sample. The method generally comprises a poly-A polymerization step or a ligation step to add a universal sequence to the 3′-end of all RNA molecules, followed by a universal primer-mediated cDNA synthesis, solid-phase selection, assay oligo annealing, extension and PCR amplification/labeling. The method of the invention can be practiced to amplify and label a small amount of miRNA or other ncRNA. The resulting amplification product can be read out on a universal array or an array with miRNA-specific or ncRNA-specific probes. The invention has multiple embodiments, including methods, compositions, and kits. In general, the nucleic acids, compositions, and kits comprise materials that are useful in carrying out the methods of the invention or are produced by the methods, and that can be used to detect small target nucleic acid sequences present in samples, in particular, small RNA species.

### BRIEF DESCRIPTION OF THE DRAWINGS

FIG. 1 shows a schematic of polyadenylation of the 3′ end of total RNA or purified small RNA, and subsequent cDNA synthesis.

FIG. 2 shows a schematic of chimera oligonucleotide linker attachment to the 3′ end of total RNA or purified small RNA, and subsequent cDNA synthesis.

FIG. 3 shows a schematic of the annealing of mi-RNA specific oligonucleotide probes to the 1^{st} strand cDNA templates and subsequent solid phase primer extension step. The cDNA templates can be obtained, for example, from the methods set forth in either FIG. 1 or 2.

FIG. 4 shows a schematic of the annealing of mi-RNA specific oligonucleotide probes and mismatch probes to the 1^{st} strand cDNA templates and subsequent solid phase primer extension step. The cDNA templates can be obtained, for example, from the methods set forth in either FIG. 1 or 2.

FIG. 5 shows validation of the method for detecting small RNAs modified at the 3′ end by either the universal linker chimera oligonucleotide (left panel) or the poly(A) oligonucleotide sequence (right panel).

FIG. 6 shows scatter plots comparing expression levels measured between technical replicates for astrocytes, H683 cells, B104.7 cells or NT21 cells. All 8 data sets were obtained using 200 ng total RNA input followed by modification, amplification and detection of miRNA on a universal array.

FIG. 7 shows scatter plots comparing expression levels measured between two liver RNA samples subjected to modification, amplification and detection of miRNA on a universal array, the first sample using 200 ng of total RNA as input and the second sample using as input microRNA enriched from 1 microgram of total RNA.

FIG. 8 shows a scatter plot indicating concordance between results obtained using modification, amplification and detection of miRNA on a universal array and results using RT-PCR.

### DETAILED DESCRIPTION OF THE INVENTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different sequence where this is logically possible. However, the sequence described below is preferred.

It must be noted that, as used in the specification and the appended claims, the singular forms “a,” “an” and “the” include plural referents unless the context clearly dictates otherwise. Thus, for example, reference to “an oligonucleotide” includes a plurality of oligonucleotides. Similarly, reference to “an RNA” includes a plurality of different identical (sequence) RNA species.

Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation. In this specification and in the claims that follow, reference will be made to a number of terms that shall be defined to have the following meanings unless a contrary intention is apparent.

“Optional” or “optionally” means that the subsequently described circumstance may or may not occur, so that the description includes instances where the circumstance occurs and instances where it does not. For example, if a step of a process is optional, it means that the step may or may not be performed, and, thus, the description includes embodiments wherein the step is performed and embodiments wherein the step is not performed (i.e. it is omitted).

The invention provides a method of detecting small target nucleotide sequences, in particular small RNA species that are present in a sample. The method generally comprises a poly-A polymerization step or a ligation step to add a universal sequence to the 3′-end of all RNA molecules, followed by a universal primer-mediated cDNA synthesis, solid-phase selection, assay oligo annealing, extension and PCR amplification/labeling. The method of the invention can be practiced to amplify and label a small amount of miRNA or other ncRNA. The resulting amplification product can be read out on a universal array or an array with miRNA-specific or ncRNA-specific probes. The invention disclosed herein has multiple embodiments, including methods, compositions, and kits. In general, the nucleic acids, compositions, and kits comprise materials that are useful in carrying out the methods of the invention or are produced by the methods, and that can be used to detect small target nucleic acid sequences present in samples.

The invention is directed, in part, to a method for determining the presence of a small target nucleotide sequence in a sample. According to particular embodiments of the method amplification and labeling are both achieved. An advantage of linking amplification and labeling in the methods is that a target nucleotide sequence that may be present in only small amounts in a sample can be amplified to a level that is readily detectable and distinguishable from other sample components. Furthermore, fairly uniform conditions can be used to faithfully and proportionally amplify a plurality of target nucleotide sequences from a sample in a multiplex format such that each target can be detected and distinguished from other targets in the sample. This method encompasses directly modifying a plurality of target nucleic acid species contained in a sample by adding the same universal priming site sequence, or same pair of universal priming site sequences, to the target nucleic acid species such that a single species of universal primer can be used for amplification of the plurality of species. A unique address sequence can be associated with each target nucleic acid species in the plurality such that one target can be distinguished from another in the plurality following amplification of the plurality with a universal primer.

The invention is described herein with regard to manipulations carried out on a particular sequence or nucleic acid having the sequence. It will be understood that several of the manipulations can produce a complementary molecule or complementary sequence. It will be further understood that several of the manipulations set forth herein can be carried out for either a first strand or its complement to achieve a similar result. For purposes of clarity and brevity, the methods are, for the most part, exemplified with respect to a single sequence. Unless explicitly indicated to the contrary, the methods and compositions described herein with regard to a particular sequence are intended to include the complement of the particular sequence.

In a preferred embodiment, the small target nucleotide sequence is an RNA sequence, for example, a miRNA. The small size of these targets can make it difficult to amplify the sequences. An advantage of the methods set forth herein is that addition of one or more universal priming sites, address sequences or a combination thereof allows small target nucleotide sequences to be amplified to levels that are convenient for a variety of detection methods.

The term “sample” as used herein relates to a material or mixture of materials, typically, although not necessarily, in fluid form, containing one or more components of interest. The term refers to a sample of tissue or fluid isolated from an individual, including but not limited to, for example, blood, plasma, serum, spinal fluid, lymph fluid, skin, respiratory, intestinal and genitourinary tracts, tears, saliva, milk, cells, tumors, organs, and also samples of in vitro cell culture constituents. Suitable sources from which the target polynucleotides are derived include, but are not limited to, cell, tissue or fluid. Different biological sources can encompass different cells/tissues/organs of the same individual, or cells/tissues/organs from different individuals of the same species, or cells/tissues/organs from different species.

A method of the invention can include a step of adding a universal priming site to a small target nucleic acid. The universal priming site can be added to the small target nucleic acid by direct modification such as ligation of a nucleic acid having the universal priming site sequence or enzyme catalyzed addition of nucleotides to form such a sequence. Exemplary enzymes that can be used to add nucleotides to form a universal priming sequence include, without limitation, a polymerase, polyadenylation polymerase or terminal transferase.

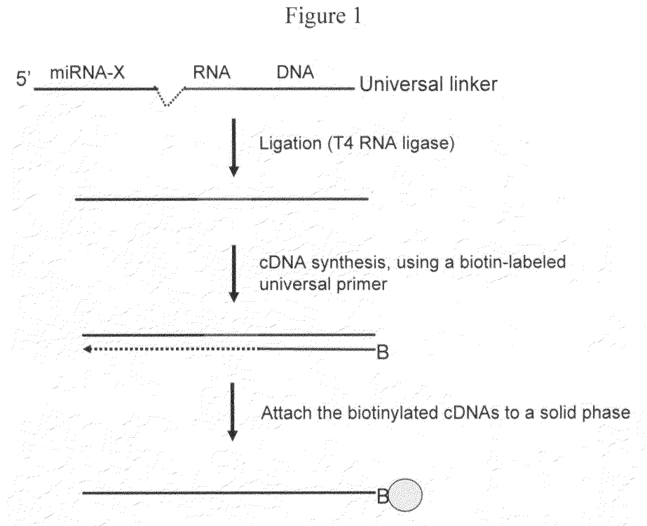

In one embodiment, a universal priming sequence is added to the 3′ end of target nucleic acids in a sample by adding a chimera nucleic acid to the 3′ end of the nucleotide species. For example and as shown in FIG. 1, a chimera nucleic acid including RNA bases at its 5′ end and DNA bases at its 3′ end can be ligated to a target RNA. The RNA bases at the 5′ end of the chimera allow an RNA ligase enzyme, such as T4 RNA ligase, to ligate the chimera to the 3′ end of the target RNA. The DNA bases at the 3′ end of the chimera serve as the universal priming site. A primer that complements the universal priming site can be used to subsequently convert the modified target RNA into complementary DNA (cDNA).

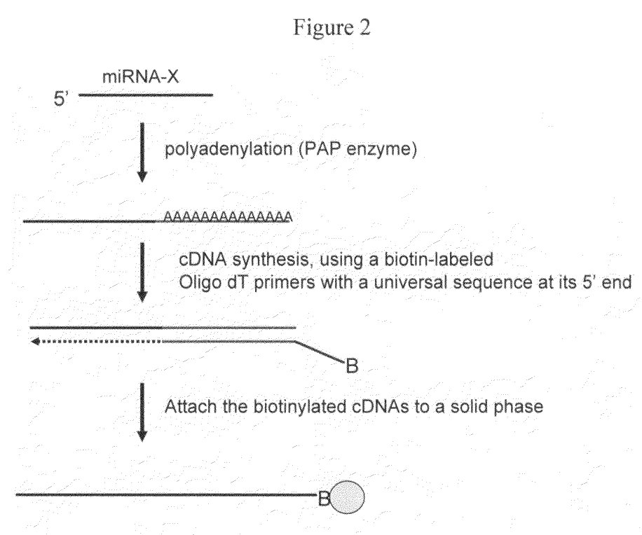

A universal priming sequence can also be added to the 3′ end of target RNAs using the method shown in FIG. 2. As shown, a target RNA species in the sample is modified by polyadenylating the 3′ end. The poly A sequence serves as a universal priming site such that a poly T primer can be used to subsequently convert the polyadenylated target nucleic acid into a complementary DNA (cDNA). Similarly terminal transferase can be used to add other homopolymeric sequences such as poly-G, -C or -T which can serve as universal priming sites.

It will be appreciated that when applied to samples having a mixture of different target nucleic acids, the methods shown in FIGS. 1 and 2 will produce a plurality of different cDNA species that, although having different target sequences, will have a common 5′ poly A sequence. A common 5′ sequence, such as the poly a sequence exemplified in the figures, can in turn be used as a universal priming site in subsequent steps such as those set forth below. It will be understood that the 3′ end of the primer used for cDNA synthesis can hybridize to the modified target nucleic acid and the primer can further include a 5′ tail that does not anneal to the target RNA. The 5′ tail of the primer will be incorporated into the cDNA product and can itself function as a universal priming site for subsequent amplification of the cDNA. Taking for example the primer used for cDNA synthesis in FIG. 2, the tail between the oligo dT region and the biotin label can include a universal priming site sequence.

Although the embodiments exemplified in FIGS. 1 and 2 do not require a template to direct activities of the ligase or polymerase, it will be understood that a template can be used. For example, the target nucleic acid can be hybridized to a template nucleic acid such that a portion of the template nucleic acid forms an overhang that serves as a template for polymerase catalyzed addition of a universal priming sequence to the 3′ end of the target nucleic acid. In this example, the overhang encodes the sequence of the universal priming site. Similarly, a target nucleic acid can be hybridized to a template nucleic acid such that a portion of the template nucleic acid forms an overhang that is complementary to a nucleic acid bearing a universal priming site sequence to direct ligation of the nucleic acid bearing a universal priming site sequence to either the 5′ or 3′ end of the target nucleic acid. The template nucleic acid can then be removed and a primer that complements the universal priming site can be used to subsequently convert the modified target RNA into complementary DNA (cDNA).

As indicated above, a method of the invention can include a step of converting a modified target nucleic acid, such as a small RNA bearing a universal priming site, into a complementary DNA (cDNA) sequence. A cDNA molecule synthesized using the methods set forth herein can include an affinity label or purification tag. The affinity label can be introduced due to its presence in a primer used for cDNA synthesis as shown for example in FIGS. 1 and 2 where the primers include a biotin. Alternatively or additionally, an affinity label or purification tag can be introduced into cDNA by incorporation of nucleotides having label or tag moieties during cDNA synthesis. Modifications can also be made post cDNA synthesis. The presence of an affinity label or purification tag can allow the cDNA to be immobilized to a solid phase support. Exemplary labels, tags and solid supports are set forth in further detail below.

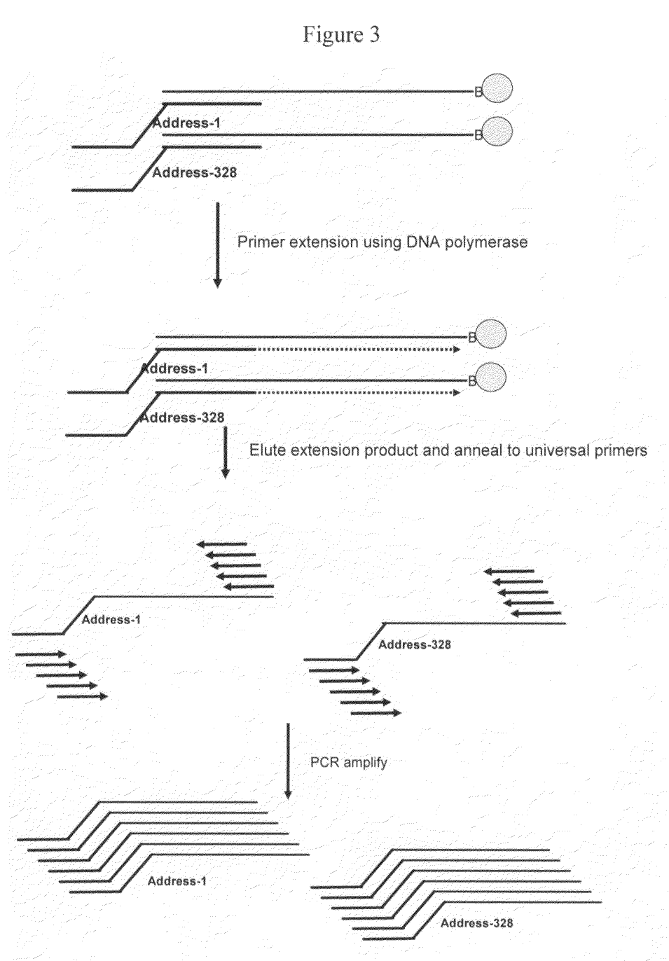

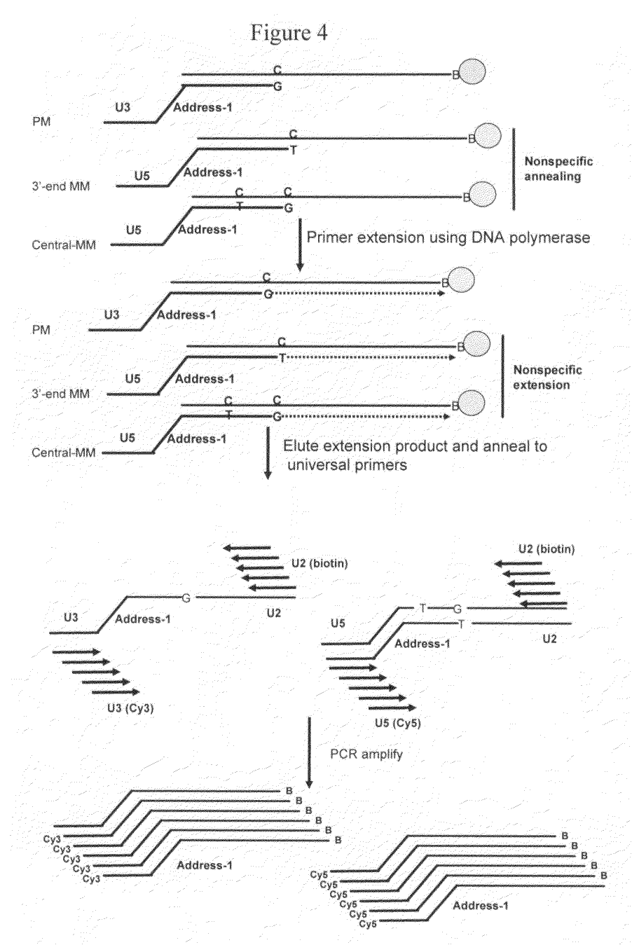

Once converted, one or more cDNA molecules can be immobilized to a solid support and contacted with one or more probe nucleic acids under conditions that allow sequence specific annealing, wherein each probe nucleic acid corresponds to a small target nucleotide sequence. Exemplary embodiments are shown in FIGS. 3 and 4. Immobilization can be mediated by an affinity label or purification tag present on the cDNA molecule such as a biotin group introduced in accordance with the examples of FIGS. 1 and 2. In a subsequent step, also exemplified by the embodiments shown in FIGS. 3 and 4, a probe nucleic acid can be extended in a manner to produce an extended probe having a sequence that is complementary to the immobilized cDNA species. The extended probe can then be removed from the immobilized cDNA species, for example, using known methods of nucleic acid denaturation. Subsequently, the extended probe can be amplified to generate an amplicon. Detection of the amplicon indicates the presence of a small target nucleotide sequence. The method is particularly useful when carried out in a multiplex format. More specifically, a plurality of the cDNA molecules, each bearing different target nucleotide sequences, can be immobilized and contacted with respective target-specific probe nucleic acids which are in turn extended and amplified such that each species of amplicon can be detected to indicate the presence of the respective target nucleotide sequence.

Immobilization of a cDNA molecule or other nucleic acid produced in accordance with the methods set forth herein provides the advantage of facilitating removal of impurities. For example, non-target nucleic acids (i.e. those not bearing a target nucleotide sequence) that are present in a sample and therefore do not obtain a particular affinity label or purification tag during a target-specific modification step can be removed since they will not have affinity for the solid support used to immobilize the labeled nucleic acids having a target sequence. It will be understood however that removal of non-target sequences is not necessarily required such as in embodiments wherein a sufficiently specific detection method is used to detect target sequences in the presence of non-target sequences. For example, in embodiments where small RNA molecules are polyadenylated and the poly A sequence exploited for immobilization, non-target RNA molecules can also be polyadenylated and immobilized. Subsequent detection conditions can be used that allow the target small RNA molecules to be detected regardless of non-target RNA molecules having been immobilized.

Another advantage of immobilizing a cDNA molecule or other nucleic acid is that it provides a means to separate them from a solution of mixture thereby facilitating concentrating the nucleic acids or transferring them to a new solution. A further advantage is that immobilization and washing allows un-hybridized and/or mis-hybridized probes to be removed prior to subsequent steps. Exemplary solid phase substrates that can be used for immobilization include, but are not limited to, those set forth below in the context of arrays.

An address sequence, universal priming sequence or both can be added to a target nucleotide sequence using a target-specific probe that hybridizes to cDNA having the target nucleic acid sequence. The embodiments exemplified in FIGS. 3 and 4, utilize a target specific probe having an address sequence and universal priming site that anneals to the cDNA such that when the probe is extended the resulting copy includes the universal priming site, the target nucleotide sequence (derived from the cDNA) and the address sequence. In the embodiment shown in the Figures, the copy will also include a second universal priming site that had been added during the cDNA synthesis step. It will be understood that a universal priming site, address sequence or both can be added to a target nucleotide sequence using other methods. For example, an address sequence, at least one universal priming site or combination thereof can be present in a pair of ligation probes. A particular sequence is considered to be present in a pair of probes if either probe contains the sequence. The two ligation probes can hybridize to the same strand of a cDNA (or other nucleic acid) bearing a target nucleotide sequence and if they are adjacent the two ligation probes can be ligated. Alternatively, if there is a gap between the ligation probes when they are hybridized to the cDNA then one can be extended and then the extended probe ligated to the other. The resulting ligation product will include the address sequence, at least one universal priming site or combination thereof and will be formed in a target specific manner. A single pre-circle probe can be used in place of the two ligation probes in such a ligation step. Such a precircle probe can include an address sequence, universal priming sequence or both. Exemplary ligation methods that can be used are described, for example, in US 2003/0108900; US 2003/0170684; US 2004/0121364; and US 2003/0215821, each of which is incorporated herein by reference. Other methods for adding address sequences and/or universal priming sites in a target-specific manner can be used, including for example, those described in these references.

It will be understood that address sequences need not be used. Instead nucleic acids having small target nucleotide sequences such as cDNA molecules, amplicons or the like can be detected based on other characteristics. For example, cDNA molecules, amplicons or the like can be hybridized to arrays having probes specific for the small target nucleotide sequences.

In accordance with the methods set forth herein a nucleic acid molecule can be synthesized that includes a target nucleotide sequence along with an address sequence or universal priming site. In particular embodiments, the nucleic acid molecule can include a target nucleotide sequence, address sequence and two universal priming sites. Exemplary nucleic acids that can be produced by a method of the invention are shown in FIGS. 3 and 4. These nucleic acids include a target nucleotide sequence and address sequence flanked by a 5′ universal priming site and 3′ universal priming site. As shown in the figures the nucleic acids can be amplified using universal primers to produces copies having the universal primers, any labels attached to the universal primers, the address sequence and the target nucleotide sequence. These amplicons can be detected using methods set forth in further detail below.

In a further embodiment, the invention provides nucleic acid species. The nucleic acid species are useful in performing at least one embodiment of the methods of the invention, or are created by at least one embodiment of the invention. The nucleic acid species thus may be extension probe oligonucleotides, amplification primers, small nucleotide sequences for use as positive controls, and other nucleic acid species that are useful for performing one or more steps of the claimed method.

An “oligonucleotide” is a molecule containing from 2 to about 100 nucleotide subunits. The term “nucleic acid” and “polynucleotide” are used interchangeably herein to describe a polymer of any length composed of nucleotides, e.g., deoxyribonucleotides or ribonucleotides, or compounds produced synthetically that can hybridize with naturally occurring nucleic acids in a sequence specific manner similar to that of two naturally occurring nucleic acids, e.g., can participate in Watson-Crick base pairing interactions. The terms “nucleoside”, “nucleotide”, “oligodeoxynucleotide”, and “deoxyribonucleotides” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. Modified nucleosides or nucleotides also include molecules having structural features that are recognized in the literature as being mimetics, derivatives, having similar properties, or other like terms, and include, for example, polynucleotides incorporating non-natural (not usually occurring in nature) nucleotides, unnatural nucleotide mimetics such as 2′-modified nucleosides, peptide nucleic acids, oligomeric nucleoside phosphonates, and any polynucleotide that has added substituent groups, such as protecting groups or linking moieties.

In general, probes of the present invention are designed to be complementary to a target sequence (either the target sequence of the sample or products derived therefrom using methods such as those described herein), such that hybridization of the target and the probes of the present invention occurs. This complementarity need not be perfect; there may be any number of base pair mismatches that will interfere with hybridization between the target sequence and the single stranded nucleic acids of the present invention. However, if the number of mismatches is so great that no hybridization can occur under even the least stringent of hybridization conditions, the sequence is not a complementary target sequence. Thus, by “substantially complementary” herein is meant that the probes are sufficiently complementary to the target sequences to hybridize under the selected reaction conditions. The relationship of probe complementarity and stringency of hybridization sufficient to achieve specificity is well known in the art and described further below in reference to sequence identity, melting temperature and hybridization conditions. Therefore, substantially complementary probes can be used in any of the detection methods of the invention. Such probes can be, for example, perfectly complementary or can contain from 1 to many mismatches so long as the hybridization conditions are sufficient to allow probe discrimination between a target sequence and a non-target sequence. Accordingly, substantially complementary probes can contain sequences ranging in percent identity from 100, 99, 98, 97, 96, 95, 94, 93, 92, 91, 90, 89, 85, 80, 75 or less.

In a further embodiment, compositions are provided. Typically, the compositions comprise one or more component that is useful for practicing at least one embodiment of the methods of the invention, or is produced through practice of at least one embodiment of the methods of the invention. The compositions thus can comprise one or more extension probe oligonucleotides according to the invention. The compositions also can comprise labeled primers complementary to the 3′ end of the modified nucleotide species. They also can comprise a universal linker consisting of a chimeric oligonucleotide as described herein. The compositions also can comprise two or more amplification primers, at least one ligase, at least one polymerase, and/or one or more detectable labels.

In an additional embodiment, kits are provided. Kits according to the invention provide at least one component that is useful for practicing at least one embodiment of the methods of the invention. Thus, a kit according to the invention can provide some or all of the components necessary to practice at least one embodiment of the method of the invention. In typical embodiments, a kit comprises at least one or more nucleic acid sequences useful for practicing the methods of the invention. In various embodiments, the kit comprises most or all of the nucleic acid sequences needed to perform at least one embodiment of the method of the invention.

The term “small target nucleotide sequence” refers to any nucleotide sequence to be detected using the methods described herein. Suitable target nucleotide sequences include, for example, DNA, cDNA, mRNA and non-coding RNA, for example, tRNA and rRNA, miRNA, pri-miRNA, short interfering RNAs (siRNAs), small nuclear RNAs (snRNAs), small nucleolar RNAs (snoRNAs), small cytoplasmic RNAs (scRNAs), tiny non-coding RNAs (tncRNA), small modulatory RNA (smRNA), package RNAs (pRNAs), guide RNAs (gRNAs), 4.5S RNA, and 6S RNA. In particular embodiments, the small nucleotide sequence can be a small RNA selected from short interfering miRNA, pri-miRNA, short interfering RNAs (siRNAs), small nuclear RNAs (snRNAs), small nucleolar RNAs (snoRNAs), small cytoplasmic RNAs (scRNAs), tiny non-coding RNAs (tncRNA), small modulatory RNA (smRNA), package RNAs (pRNAs), guide RNAs (gRNAs), 4.5S RNA, and 6S RNA, or combinations thereof. See Novina et al., Nature 430: 161-164 (2004). In particular embodiments, small RNAs may be at least about 4 bases long, at least about 6 bases long, at least about 8 bases long, or longer. The invention can be advantageously utilized for small target nucleic acid sequences, which can be less than 50, 45, 40, 36, 30, 25, 20, 15, or 10 nucleotides in length. The methods of the invention are particularly suitable for detection of small nucleotide sequences, in particular small RNA sequences such as non-coding RNA. In particular embodiments, the small target nucleotide sequences encompass micro RNA (miRNA). As used herein, miRNA are those molecules that meet the criteria of the Sanger Institute miRNA Registry (and precursors to those molecules). Thus, this embodiment of the invention provides methods for determining the presence or absence of miRNA molecules in a sample. The methods of the invention can be practiced, for example, to detect miRNA sequences less than 30 nucleotides, less than 28 nucleotides, less than 26 nucleotides, less than 24 nucleotides, less than 22 nucleotides, less than 20 nucleotides, less than 18 nucleotides, less than 16 nucleotides, less than 15 nucleotides or smaller. In some embodiments, a miRNA target sequence is a variant of a miRNA. Micro RNAs are reviewed, for example, in Ambros, Nature (2004) 431:350-5; Tang, Trends Biochem Sci (2005) 30:106-114; and Bengert and Dandekar, Brief Bioinform. (2005) 6:72-85.

The term “siRNAs” refers to short interfering RNAs. In some embodiments, siRNAs comprise a duplex, or double-stranded region, where each strand of the double-stranded region is about 18 to 25 nucleotides long; the double-stranded region can be as short as 16, and as long as 29, base pairs long, where the length is determined by the antisense strand. Short interfering RNA is reviewed, for example, in Jones, et al., Curr. Opin. Pharmacol. (2004) 4:522-7; and in Tang, supra (2005).

The methods of the invention can be practiced with unpurified samples containing total nucleotide species such as crude cell lysates or can include an optional initial step of enriching the sample for the nucleotide species of interest. A surprising advantage of the current methods is that very small or degraded targets can be detected, in particular, for samples containing less than 500 ng, less than 400 ng, less than 300 ng, less than 200 ng, less than 150 ng, less than 100 ng, less than 75 ng, less than 50 ng, less than 40 ng, less than 30 ng of total RNA. The sample of RNA may be obtained from any source. For example, the sample of RNA may be any RNA sample, typically a sample containing RNA that has been isolated from a biological source, e.g. any plant, animal, yeast, bacterial, or viral source, or a non-biological source, e.g. chemically synthesized. In particular embodiments, the sample of RNA includes one or more small RNAs, such as, for example, microRNAs (miRNA), tiny non-coding RNAs (tncRNA) and small modulatory RNA (smRNA). In particular embodiments, the sample includes isolated small RNAs, for example, the sample results from an isolation protocol for small RNA. In certain embodiments, the small RNA targets may include isolated miRNAs, such as those described in the literature and in the public database. In particular embodiments, the sample includes isolated small RNAs, for example, the sample results from an isolation protocol for small RNA, especially RNAs less than about 500 bases long, for example, less than about 400 bases long, less than about 300 bases long, less than about 200 bases long, less than about 100 bases long, or less than about 50 bases long. In some embodiments, the sample of RNA may be a whole RNA fraction isolated from a biological source and includes messenger RNA and small RNA. Such samples including a diverse set of RNAs, such as a whole RNA fraction, may be referenced herein as “complex” RNA samples. Such samples can include DNA or can substantially exclude DNA as desired to suit a particular application of the invention.

The methods of the invention can be performed using archived tissue samples that have been obtained from a source and preserved. Preferred methods of preservation include, but are not limited to paraffin embedding, ethanol fixation and formalin (including formaldehyde and other derivatives) fixation as are known in the art. The sample may be temporally “old”, e.g. months or years old, or just fixed. For example, post-surgical procedures generally include a fixation step on excised tissue for histological analysis. The invention methods can be practiced with the target sequence contained in the archived sample or can be practiced with target sequences that have been physically separated from the archived sample prior to performing a method of the invention.

Suitable tissue samples include, but are not limited to, bodily fluids (including, but not limited to, blood, urine, serum, lymph, saliva, anal and vaginal secretions, perspiration and semen, of virtually any organism, with mammalian samples being preferred and human samples being particularly preferred). In a preferred embodiment, the sample is a diseased tissue sample, particularly a cancer tissue, including primary and secondary tumor tissues as well as lymph node tissue and metastatic tissue. Thus, as defined herein, an archived sample can be heterogeneous and encompass more than one cell or tissue type, for example, tumor and non-tumor tissue. Preferred archived samples include solid tumor samples including, but not limited to, tumors of the brain, bone, heart, breast, ovaries, prostate, uterus, spleen, pancreas, liver, kidneys, bladder, stomach and muscle. In a preferred embodiment, the tissue sample is one for which patient history and outcome is known, such as prognostic data.

If required, the target sequence is prepared using known techniques. For example, the sample may be treated to lyse the cells, using known lysis buffers, sonication, electroporation, etc., with purification and amplification as outlined below occurring as needed, as will be appreciated by those in the art. In addition, the reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents which may be included in the assays. These include reagents like salts, buffers, neutral proteins, for example, albumin, detergents, etc., which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target.

In certain embodiment, a sample can be enriched for miRNA species using commercially available kits, for example, PureLink™ (Invitrogen). “Enriched” or “purified” generally refers to isolation of a substance (compound, polynucleotide, protein, polypeptide, polypeptide, chromosome, etc.) such that the substance constitutes a substantial portion of the sample in which it resides (excluding solvents), i.e. the relative amount of the substance to one or more other impurity is greater than in its natural or un-isolated state. Typically, a substantial portion of the sample comprises at least about 2%, at least about 5%, at least about 10%, at least about 20%, at least about 30%, at least about 50%, at least about 80%, or at least about 90% of the sample (excluding solvents). For example, a sample of isolated RNA will typically comprise at least about 2% total RNA, or at least about 5% total RNA, where percent is calculated in this context as mass (for example, in micrograms) of total RNA in the sample divided by mass (e.g. in micrograms) of the sum of (total RNA plus other constituents in the sample (excluding solvent)). Techniques for purifying polynucleotides and polypeptides of interest are well known in the art and include, for example, gel electrophoresis, ion-exchange chromatography, affinity chromatography, and sedimentation according to density. Further methods that can be used to enrich for small RNA species are described in US 2006/0019258, which is incorporated herein by reference.

In particular embodiments, the small nucleotide species contained in the sample are modified by attaching a universal oligonucleotide sequence to the 3′ end, wherein the oligonucleotide sequence is a poly(A) linker. The polyadenylation step can add at least 5 nucleotides, at least 10 nucleotides, at least 15 nucleotides, at least 20 nucleotides, at least 25 nucleotides, at least 30 nucleotides, at least 35 nucleotides or more, at least 40 nucleotides or more, at least 45 nucleotides or more, at least 50 nucleotides or more, at least 55 nucleotides or more, at least 60 nucleotides or more, at least 65 nucleotides or more, at least 70 nucleotides or more, at least 75 nucleotides or more, at least 80 nucleotides or more, at least 85 nucleotides or more, at least 90 nucleotides or more, at least 95 nucleotides or more, at least 100 nucleotides or more, at least 105 nucleotides or more, at least 110 nucleotides or more, at least 115 nucleotides or more, at least 120 nucleotides or more, at least 125 nucleotides or more, at least 130 nucleotides or more. In particular embodiments, purified small nucleotide sequences, including miRNA species, are polyadenylated by adding between 20 and 150 nucleotides. Generally, at least 18 nucleotides are added in the polyadenylation step. Once modified by attachment of the oligonucleotide sequence, the small nucleotide sequences can be converted into cDNA utilizing a primer that is complementary to the 3′ end universal oligonucleotide, for example, the poly(A) tail of the modified nucleic acid sequence. Polyadenylation can be carried out using methods known in the art such as those utilizing polyadenylation polymerase (PAP). Commercially available kits for polyadenylation can be used such as the Poly(A) tailing kit from Ambion (Austin, Tex.) or the A-Plus™ Poly(A) Polymerase Tailing Kit from Epicentre Biotechnologies (Madison, Wis.).

In accordance with the methods set forth herein, small nucleotide species contained in a sample can be modified by attaching a universal priming sequence to the 3′ end via use of a 5′ phosphorylated chimera nucleic acid. The term “chimera nucleic acid” refers to a nucleic acid or oligonucleotide having RNA bases at its 5′ end followed by DNA bases at its 3′ end. In particular embodiments the 5′ end RNA bases can be 10 or less, 9 or less, 8 or less, 7 or less, 6 or less, 5 or less, 4 or less, 3 or less, 2 or less. In a further embodiment, the '5 end RNA bases can be between 2 and 10 bases, between 3 and 8 bases, between 3 and 7 bases, between 3 and 5 bases, between 4 and 7 bases, between 4 and 6 bases. In addition, the DNA bases that follow at the 3′ end of the RNA bases can be 30 or less, 29 or less, 28 or less, 27 or less, 26 or less, 25 or less, 24 or less, 23 or less, 22 or less, 21 or less, 20 or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, 4 or less, 3 or less, 2 or less. In a further embodiment, the DNA bases can range between 2 and 30 bases, 3 and 25 bases, 3 and 20 bases, 3 and 15 bases, 3 and 10 bases, 5 and 30 bases, 5 and 25 bases, 5 and 20 bases, 5 and 15 bases, 5 and 10 bases, 10 and 30 bases, 10 and 20 bases, 10 and 15 bases, 15 and 30 bases, 15 and 25 bases, 15 and 19 bases, 15 and 18 bases, 15 and 17 bases, 15 and 20 bases. Once modified by attachment of the universal linker, the small nucleotide sequences can be converted into cDNA utilizing a primer that is complementary to a part or all of the 3′ end universal oligonucleotide sequence.

As described above, in the various embodiments, a cDNA sequence can be obtained using a labeled primer complementary to the 3′ end of the modified nucleotide species. As described further below, the label can comprise biotin. The cDNA can be obtained by any methods described below, for example, using reverse transcriptase (RT). By “reverse transcriptase” or “RNA-directed DNA polymerase” herein is meant an enzyme capable of synthesizing DNA from a DNA primer and an RNA template. Suitable RNA-directed DNA polymerases include, but are not limited to, avian myloblastosis virus reverse transcriptase (“AMV RT”) and the Moloney murine leukemia virus RT. In one embodiment, thermo-stable reverse transcriptase is preferred because the cDNA can be obtained at a high temperature so as to allow opening up of secondary structures associated with small RNA species, for example, stem/loop formations. The cDNA sequence can subsequently be immobilized to a solid support.

Once immobilized, cDNA sequences can be contacted with a pool of target specific probe oligonucleotides under conditions that allow sequence specific annealing. In order to minimize non-specific annealing, analytical variables such as priming, temperature and time of primer annealing, primer extension and denaturation, as well as the concentrations of magnesium chloride, Taq polymerase, deoxynucleotide triphosphate, primers and BSA can be optimized by the user. If desired, a temperature gradient processing can be performed from a high temperature point to a low temperature point. Furthermore, by adding endonuclease into the reaction, a non-paired base pair portion that is a non-complementary portion contained in a complementary double strand is recognized, cleaved, and eliminated. By this processing, it is possible to measure and detect only a portion forming a completely complementary double strand. Moreover, by combining the enzyme treatment step with the temperature gradient processing, it is possible to minimize non-specific annealing. The endonuclease can be any endonuclease that recognizes and cleaves a non-complementary nucleic acid portion in a complementary double strand. Examples of an endonuclease preferably used include a DNA repair enzyme such as uvrABC exonuclease. Solid phase second stand extension can subsequently be performed according to any methods desired by the user and as described further below.

As disclosed herein, each target specific probe nucleic acid can correspond to a small target nucleotide sequence that can be present in an immobilized cDNA. Once annealed to an immobilized cDNA, non-hybridized probes can be removed by a stringent wash while annealed probes can be extended in a manner to produce a nucleic acid product having a sequence that is complementary to the cDNA. In one embodiment, the target specific probe nucleic acids can include a unique address sequence and a universal priming sequence. As described in more detail below, the use of address sequences and universal priming sequences provides several advantages for multiplex detection of small target nucleotide sequences.

In a preferred embodiment, a probe includes an address sequence, (sometimes referred to as an “adapter sequence,” “zip code” or “bar code”). Address sequences facilitate immobilization of probes, or amplicons thereof, to “universal arrays”. That is, arrays contain capture probes that are not necessarily target sequence specific, but rather specific to individual (preferably) address sequences. Thus, an “address sequence” is a nucleic acid that is generally not native to the target sequence, i.e. is exogenous, but is added or attached to the target sequence. It should be noted that in this context, the target sequence can include the primary sample target sequence, or can be a derivative target such as a reactant or product of the reactions outlined herein; thus for example, the target sequence can be a PCR product, a probe extension product or a ligated probe, etc.

One preferred form of address sequences are hybridization adapters. In this embodiment adapters are chosen so as to allow hybridization to the complementary capture probes on a surface of an array. Adapters serve as unique identifiers of the probe and thus of the target sequence. In general, sets of address sequences and the corresponding capture probes on arrays are developed to minimize cross-hybridization with both each other and other components of the reaction mixtures, including the target sequences and sequences on the larger nucleic acid sequences outside of the target sequences (for example, to sequences within genomic DNA or mRNA). Other forms of adapters are those that have characteristic mass, charge or charge to mass ratio such that they can be used as mass tags that can be separated using mass spectroscopy, electrophoretic tags that can be separated based on electrophoretic mobility, etc. Some adapter sequences are outlined in US 2003/0096239, hereby incorporated by reference. Preferred adapters are those that are not found in a genome, preferably a human genome, and they do not have undesirable structures, such as hairpin loops.

As set forth in further detail below, a target sequence can be identified according to the presence of a target specific probe having the address sequence. Furthermore, two target sequences having different address sequences can be identified and distinguished from each other according to the locations of the respective target-specific probes (or amplicons derived therefrom) at known locations on a universal array. An exemplary method in which target sequences are distinguished based on differences in address sequences is described below in the context of FIG. 3. A target sequence can also be identified according to characteristics of a particular label associated with a target specific probe. For example, two target sequences having the same address sequence but different associated labels can be identified and distinguished from each other according to detection of the different labels of the respective target-specific probes (or amplicons derived therefrom) at one or more known locations on a universal array. An exemplary method in which target sequences are distinguished based on differences in address sequences and differences in label characteristics is described below in the context of FIG. 4.

As will be appreciated by those in the art, the attachment, or joining, of the address sequence to the target sequence can be done in a variety of ways. In a preferred embodiment, the address sequences are added to the primers of the reaction (extension primers, amplification primers, etc.) during the chemical synthesis of the primers. The address sequence then gets added to the reaction product during the reaction; for example, the primer gets extended using a polymerase to form the new target sequence that now contains the address sequence. Alternatively, the address sequences can be added enzymatically. Furthermore, the address can be attached to the target after synthesis; this post-synthesis attachment can be either covalent or non-covalent. As will be appreciated by those in the art, the address sequence can be attached either on the 3′ or 5′ ends, or in an internal position, depending on the configuration of the system.

An address sequence can be one that is not found in a particular organism such as a mammal, primate, human, nonhuman primate. Thus, an address sequence can be chosen to prevent hybridization to nucleic acids in the mRNA of an organism for which non-mRNA sequences are to be identified.

In one embodiment the use of address sequences allow the creation of more “universal” surfaces; that is, one standard array, comprising a finite set of capture probes can be made and used in any application. The end-user can customize the array by designing different soluble target probes, which, as will be appreciated by those in the art, is generally simpler and less costly than designing and creating different arrays for different target sequences. In a preferred embodiment, an array of different and usually artificial capture probes are made; that is, the capture probes do not have complementarity to known target sequences. The address sequences can then be incorporated in the target probes or other nucleic acids bearing the target sequences.

As will be appreciated by those in the art, the length of the address sequences will vary, depending on the desired “strength” of binding and the number of different address desired. In a preferred embodiment, address sequences range from about 6 to about 500 basepairs in length, with from about 8 to about 100 being preferred, and from about 10 to about 25 being particularly preferred.

A nucleic acid useful in the methods set forth herein can be constructed so as to contain the necessary priming site or sites for a subsequent amplification step. In a preferred embodiment the priming sites are universal priming sites. In a preferred embodiment, one universal priming sequence or site is used. In this embodiment, a preferred universal priming sequence is the RNA polymerase T7 sequence, that allows the T7 RNA polymerase to make RNA copies of the nucleic acid. Additional disclosure regarding the use of T7 RNA polymerase is found in U.S. Pat. Nos. 6,291,170, 5,891,636, 5,716,785, 5,545,522, 5,922,553, 6,225,060 and 5,514,545, all of which are expressly incorporated herein by reference. Poly A is another particularly useful universal priming site.

In a preferred embodiment, for example when amplification methods requiring two primers such as PCR are used, each nucleic acid preferably comprises an upstream universal priming site (UUP) and a downstream universal priming site (DUP). Again, “upstream” and “downstream” are not meant to necessarily limit to a particular 5′-3′ orientation, and will depend on the orientation of the system. Preferably, only a single UUP sequence and a single DUP sequence is used in a nucleic acid or probe set, although as will be appreciated by those in the art, different assays or different multiplexing analysis may utilize a plurality of universal priming sequences. In some embodiments nucleic acids may comprise different sets of universal priming sequences. In addition, the universal priming sites are preferably located at the 5′ and 3′ termini of a nucleic acid, as only sequences flanked by priming sequences will be amplified.

In addition, universal priming sequences are generally chosen to be as unique as possible given the particular assays and host genomes to ensure specificity of the assay. As will be appreciated by those in the art, in general, highly multiplexed reactions can be performed, with all of the universal priming sites being the same for all reactions. Thus, a single species of universal primer can be used to copy or amplify all of the target nucleic acids in the multiplex reaction. Alternatively, “sets” of universal priming sites and corresponding probes can be used, either simultaneously or sequentially. Accordingly, a universal priming sequence (or pair of universal priming sequences) is common to a subset of two or more target nucleic acids in a multiplex reaction. The multiplex reaction can include several subsets of target nucleic acids, each subset having a different universal priming site. For example, sets of priming sequences/primers may be used; that is, one reaction may utilize 500 different target probes with a common first priming sequence, and an additional 500 different probes with a second common priming sequence, wherein the first priming sequence differs from the second priming sequence. Thus, several universal primers can be used to copy or amplify the different subsets of target nucleic acid molecules in a single multiplex reaction.

As will be appreciated by those in the art, when two priming sequences are used for PCR amplification, the orientation of the two priming sites is generally different. That is, one PCR primer will directly hybridize to the first priming site, while the other PCR primer will hybridize to a second priming site on the complementary strand. Stated differently, the first priming site is in sense orientation, and the second priming site is in antisense orientation.

Target specific probe nucleic acids, once hybridized to target cDNAs, can be contacted with an enzyme such as a polymerase in the presence of nucleotides to form extended target specific probe nucleic acids. The extended target specific probe nucleic acids are then eluted from the immobilized cDNA species, and contacted with amplification primers to form amplicons. In multiplex embodiments the amplification primers are universal primers. In one embodiment the eluted product is purified by binding to a binding partner for the affinity tag. Then the purified and modified product can be contacted with the amplification primers for amplification, forming amplicons. The amplicons are then detected as an indication of the presence of the particular target nucleotide sequence.

In a preferred embodiment, the target specific probe nucleic acid includes from 5′ to 3′, a universal priming site, a unique address sequence, and a target specific sequence. Priming sequences hybridize with amplification primers; the adapter sequence mediates attachment of the amplicons to a support for subsequent detection of amplicons. In multiplex embodiments, as described herein, the priming site sequences can be universal priming site sequences.

Detection of different target sequences in a multiplex format can proceed on a number of levels. For example, as shown in FIG. 3, a unique address sequence present on a target-specific probe can be distinct for a particular target sequence such that detection of the address indicates presence of the target sequence. As shown in FIG. 3, following amplification of the unique addresses with labeled primers and hybridization of the amplicons to a universal array, detection of the address indicates presence of the particular target nucleotide sequence to be detected. In multiplex embodiments, target-specific probes having different address sequences (i.e. address-1 and address-328) can be distinguished according to their locations on a universal array. Alternatively and as shown in FIG. 4, two different target specific probes can have the same address sequence but different universal priming sites (i.e. U3 and U5) such that the two target sequences can be distinguished based on the different labels attached to different universal primers (Cy3 on the U3 primer and Cy5 on the U5 primer) used in the amplification step. Higher level multiplexing can be achieved for the embodiment shown in FIG. 4, by using multiple addresses in addition to multiple labels. For example in a sample having several different loci some of which have heterozygous alleles present, the different loci can be distinguished based on array location and the different alleles at each locus can be distinguished based on which of the two fluorophores is present at a particular array location.

In particular embodiments, a multiplex PCR reaction is performed using universal primers as described herein. That is, universal PCR primers hybridized to universal priming sites on the target sequence and thereby amplify a plurality of target sequences. This embodiment is useful because it requires only a limited number of PCR primers. That is, as few as one primer pair can amplify a plurality of target sequences.

The polymerase chain reaction (PCR) is widely used and described, and involves the use of primer extension combined with thermal cycling to amplify a target sequence; see U.S. Pat. Nos. 4,683,195 and 4,683,202, and PCR Essential Data, J. W. Wiley & sons, Ed. C.R. Newton, 1995, all of which are incorporated by reference. In general, PCR may be briefly described as follows. A double stranded target nucleic acid is denatured, generally by raising the temperature, and then cooled in the presence of an excess of a PCR primer, which then hybridizes to the first target strand. A DNA polymerase then acts to extend the primer with dNTPs, resulting in the synthesis of a new strand forming a hybridization complex. The sample is then heated again, to disassociate the hybridization complex, and the process is repeated. By using a second PCR primer for the complementary target strand, rapid and exponential amplification occurs. Thus PCR steps are denaturation, annealing and extension. The particulars of PCR are well known, and include the use of a thermostable polymerase such as Taq I polymerase and thermal cycling.

Accordingly, the PCR reaction requires at least one PCR primer, a polymerase, and a set of dNTPs. As outlined herein, the primers may comprise a label, or one or more of the dNTPs may comprise a label or both can be labeled.

While the invention methods are generally directed to PCR systems, other amplification systems can be used, as are generally outlined in U.S. Pat. No. 6,355,431, or 2005/0181394 each of which is incorporated herein by reference. Particularly useful methods are those that are carried out under isothermal conditions without the use of thermocycling.

Given the teachings and guidance provided herein, any of the compositions, methods, configurations and formats described above or below can be used in conjunction with, or in the alternative, to each other for detecting one or more target sequences in a sample or even to detect the relative amounts of two or more target sequences in a sample. Such compositions, methods, configurations and formats include, for example, the various probe and primer configurations, amplification reactions, detection systems and assay formats, including multiplexing target sequence detection.

A method of detecting relative amounts of two or more small target nucleotide sequences can utilize linear amplification as an accurate indicator of the initial relative abundance following amplification. In this regard, linear amplification maintains proportional differences between two or more sequences and avoids enhancement of biases that can result during exponential amplification due to sequence and template differences.

Linear amplification can be performed, for example, by unidirectional amplification using enzymatic polymerization as described previously. Unidirectional amplification can be performed by priming and polymerase directed extension from a single strand. The priming and extension can initiate, for example, from either strand such that there is a net increase of about one completed extension product for each round of priming. In one aspect, linear amplification includes in vitro transcription of a target sequence by polymerase extension from a promoter site. The resulting amplification level of the amplicon is directly proportional to the number of times a target sequence template is primed and extended. Linear amplification can be contrasted to exponential amplification such as PCR or rolling circle amplification where two or more extension products are formed for each round of priming typically from both complements of double stranded sequence.

The present invention particularly draws on methodologies outlined in US 2003/0215821; US 2004/0018491; US 2003/0036064; US 2003/0211489, each of which is expressly incorporated by reference in their entirety. In addition, universal priming methods are described in detail in US 2002/0006617; US 2002/0132241, each of which is expressly incorporated herein by reference. In addition, multiplex methods are described in detail US 2003/0211489; US 2003/0108900, each of which is expressly incorporated herein by reference.

A method of the invention can include a step of immobilizing a nucleic acid or detecting a small target nucleotide sequence on a solid phase substrate. A “substrate” or “solid support” useful in the invention can be any material that is appropriate for or can be modified to be appropriate for the attachment of a nucleic acid such as a nucleic acid having a target sequence. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, (and Teflon™), polysaccharides, nylon or nitrocellulose, ceramics, resins, silica or silica-based materials including silicon and modified silicon, carbon, metals, inorganic glasses, optical fiber bundles, and a variety of other polymers. Magnetic beads and high throughput microtiter plates are particularly preferred.

The composition and geometry of the solid support vary with its use. In particular embodiments, supports comprising microspheres or beads are preferred. By “microspheres” or “beads” or grammatical equivalents herein is meant small discrete particles. The composition of the beads will vary, depending on the class of bioactive agent and the method of synthesis. Suitable bead compositions include those used in peptide, nucleic acid and organic moiety synthesis, including, but not limited to, plastics, ceramics, glass, controlled pore glass (CPG) polystyrene, methylstyrene, acrylic polymers, paramagnetic materials, thoria sol, carbon graphited, titanium dioxide, latex or cross-linked dextrans such as Sepharose, cellulose, nylon, cross-linked micelles and teflon, as well as any other materials outlined herein for solid supports may all be used. “Microsphere Detection Guide” from Bangs Laboratories, Fishers Ind. is a helpful guide. Preferably, in this embodiment, when complexity reduction is performed, the microspheres are magnetic microspheres or beads. The beads need not be spherical; irregular particles may be used. In addition, the beads may be porous, thus increasing the surface area of the bead available for assay. The bead sizes range from nanometers, i.e. 100 nm, to millimeters, i.e. 1 mm, with beads from about 0.2 micron to about 200 microns being preferred, and from about 0.5 to about 5 micron being particularly preferred, although in some embodiments smaller beads may be used.

A target sequence, probe or primer can be attached to a solid support in a number of ways. In a preferred embodiment, purification tags are used. By “purification tag” herein is meant a moiety which can be used to purify a strand of nucleic acid, usually via attachment to a solid support as outlined herein. Suitable purification tags include members of binding partner pairs. For example, the tag may be a hapten or antigen, which will bind its binding partner. In a preferred embodiment, the binding partner can be attached to a solid support as depicted herein. For example, suitable binding partner pairs include, but are not limited to: antigens (such as proteins (including peptides)) and antibodies (including fragments thereof (FAbs, etc.)); proteins and small molecules, including biotin and streptavidin or avidin, enzymes and substrates or inhibitors, lectin and carbohydrates, biotin avidin; other protein-protein interacting pairs; receptor-ligands; and carbohydrates and their binding partners. Nucleic acid—nucleic acid binding pairs are also useful. In general, the smaller of the pair is attached to an NTP or primer. Preferred binding partner pairs include, but are not limited to, biotin (or imino-biotin) and streptavidin, digeoxinin and Abs, and Prolinx™ reagents (see www.prolinxinc.com/ie4/home.hmtl).

In particular embodiments, microspheres or beads can be arrayed or otherwise spatially distinguished. Exemplary bead-based arrays that can be used in the invention include, without limitation, those in which beads are associated with a solid support such as those described in U.S. Pat. No. 6,355,431 B1, US 2002/0102578 and PCT Publication No. WO 00/63437. Beads can be located at discrete locations, such as wells, on a solid-phase support, whereby each location accommodates a single bead. Alternatively, discrete locations where beads reside can each include a plurality of beads as described in, for example, U.S. patent application Nos. US 2004/0263923, US 2004/0233485, US 2004/0132205 or US 2004/0125424. Beads can be associated with discrete locations via covalent bonds or non-covalent interactions such as gravity, magnetism, ionic forces, van der Waals forces, hydrophobicity or hydrophilicity. However, the sites of an array of the invention need not be discrete sites. For example, it is possible to use a uniform surface of adhesive or chemical functionalities that allows the attachment of particles at any position. Thus, the surface of an array substrate can be modified to allow attachment or association of microspheres at individual sites, whether or not those sites are contiguous or non-contiguous with other sites. Thus, the surface of a substrate can be modified to form discrete sites such that only a single bead is associated with the site or, alternatively, the surface can be modified such that a plurality of beads populates each site.

Beads or other particles can be loaded onto array supports using methods known in the art such as those described, for example, in U.S. Pat. No. 6,355,431. In some embodiments, for example when chemical attachment is done, particles can be attached to a support in a non-random or ordered process. For example, using photoactivatible attachment linkers or photoactivatible adhesives or masks, selected sites on an array support can be sequentially activated for attachment, such that defined populations of particles are laid down at defined positions when exposed to the activated array substrate. Alternatively, particles can be randomly deposited on a substrate. In embodiments where the placement of probes is random, a coding or decoding system can be used to localize and/or identify the probes at each location in the array. This can be done in any of a variety of ways, for example, as described in U.S. Pat. No. 6,355,431; U.S. Pat. No. 7,033,754; US 2006/0073513 or WO 03/002979. A further encoding system that is useful in the invention is the use of diffraction gratings as described, for example, in US Pat. App. Nos. US 2004/0263923, US 2004/0233485, US 2004/0132205, or US 2004/0125424.

An array of beads useful in the invention can also be in a fluid format such as a fluid stream of a flow cytometer or similar device. Exemplary formats that can be used in the invention to distinguish beads in a fluid sample using microfluidic devices are described, for example, in U.S. Pat. No. 6,524,793. Commercially available fluid formats for distinguishing beads include, for example, those used in XMAP™ technologies from Luminex or MPSS™ methods from Lynx Therapeutics.

Any of a variety of arrays known in the art can be used in the present invention. For example, arrays that are useful in the invention can be non-bead-based. A useful array is an Affymetrix™ GeneChip™ array. GeneChip™ arrays can be synthesized in accordance with techniques sometimes referred to as VLSIPS™ (Very Large Scale Immobilized Polymer Synthesis) technologies. Some aspects of VLSIPS™ and other microarray and polymer (including polypeptide) array manufacturing methods and techniques have been described in U.S. Ser. No. 09/536,841, International Publication No. WO 00/58516; U.S. Pat. Nos. 5,143,854, 5,242,974, 5,252,743, 5,324,633, 5,445,934, 5,744,305, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846, 6,022,963, 6,083,697, 6,291,183, 6,309,831 and 6,428,752; and in PCT Applications Nos. PCT/US99/00730 (International Publication No. WO 99/36760) and PCT/US01/04285. Such arrays can hold over 500,000 probe locations, or features, within a mere 1.28 square centimeters. The resulting probes are typically 25 nucleotides in length.

A spotted array also can be used in a method of the invention. An exemplary spotted array is a CodeLink™ Array available from General Electric (acquired from Amersham Biosciences). CodeLink™ Activated Slides are coated with a long-chain, hydrophilic polymer containing amine-reactive groups. This polymer is covalently crosslinked to itself and to the surface of the slide. Probe or other nucleic acid attachment can be accomplished through covalent interaction between the amine-modified 5′ end of the oligonucleotide probe and the amine reactive groups present in the polymer. Probes or other nucleic acids can be attached at discrete locations (i.e. features or substrate elements) using spotting pens. Such pens can be used to create features having a spot diameter of, for example, about 140-160 microns. In a specific embodiment, nucleic acid probes at each spotted feature can be 30 nucleotides long.

Another array that is useful in the invention is one manufactured using inkjet printing methods such as SurePrint™ Technology available from Agilent Technologies. Such methods can be used to synthesize probes or other nucleic acids in situ or to attach presynthesized nucleic acids having moieties that are reactive with a substrate surface. A printed microarray can contain about 22,575 features on a surface having standard slide dimensions (about 1 inch by 3 inches). Generally, the printed nucleic acids are 25 or 60 nucleotides in length. Also useful are arrays manufactured by Nimblegen (Reykjavik, Iceland) or by Xeotron methods (available from Invitrogen, Carlsbad, Calif.).

It will be understood that the specific synthetic methods and probe or other nucleic acid lengths described above for different commercially available arrays are merely exemplary. Similar arrays can be made using modifications of the methods, and nucleic acids having other lengths can also be placed at each feature of the array.

A nucleic acid useful in the invention can include a detection label. A variety of detectible labels can be used in the methods of the invention to determine the presence or absence of one or more target nucleic acids within a population of nucleic acids and/or to determine the nucleotide sequence at one or more positions within one or more target nucleic acids within a population of nucleic acids. Different labels contained in a mixture for concurrent and/or sequential detection are selected to produce distinct signals that can be differentiated in a method of the invention. Distinctness can be accomplished by, for example, employing labels producing the same or different type of signal. For example, a set of labels where all emit fluorescent signals can be employed as the type of label. The signals can be distinguished where each label within the set emits a different colored wavelength. Similarly, a set can include different types of labels where some or all generate different types, and therefore, distinct of signals. For example, a set can be generated where one or more labels are fluorescent and one or more labels are luminescent, reflectance and/or radioactive.

A “detection label” or “detectable label” can include any moiety that allows detection. Detection labels may be primary labels (i.e. directly detectable) or secondary labels (indirectly detectable). In a preferred embodiment, the detection label is a primary label. A primary label is one that can be directly detected, such as a fluorophore. Examples of primary labels which are useful for detection and which can be combined into a set of distinct labels include, for example, fluorophores, radiolabels, quantum dots, chromophores, enzymes, affinity ligands, electromagnetic spin moieties, heavy atoms, nanoparticle light scattering labels or other nanoparticles or spherical shells and labels having any other signal generation known to those of skill in the art. Specific examples of a variety of fluorescent labels having distinct wavelengths are described further below.

Particularly useful fluorescent labels include, for example, FAM, Alexa555, Alex 647 and Alexa 750 (all from Invitrogen Corp., San Diego, Calif.). Each of these labels has an emission wavelength distinguishable from the other and therefore, can be used in a common detection mixture to distinguish individual species in the mixture. For example, FAM has an excitation wavelength of 488λ and an emission wavelength of 505λ, which is in the visible green light of the electromagnetic spectrum (˜490-540λ). Alexa555 has an excitation wavelength of 555λ and an emission wavelength of 565λ, which is in the red-orange region of the visible light spectrum (˜565-605λ). Alexa647 has an excitation wavelength of 650λ and emits at 668λ in the far-red region of the visible spectrum (˜645-670λ) whereas Alexa750 is excited at 749λ and emits at 775λ in the near-infrared region of the electromagnetic spectrum (˜685-780λ).

Fluorescent labels emitting signals in any region of the electromagnetic spectrum other than those exemplified above also can be used in the methods of the invention to generate sets of labels emitting different and distinguishable signals. Such fluorescent labels having emission wavelengths in any of the visible wavelengths of light include, for example, wavelengths ranging from visible violet light having a wavelength at about 400 nm, indigo light having a wavelength of about 445 nm, blue light having a wavelength of about 475 nm, green light having a wavelength of about 510 nm, yellow light having a wavelength of about 570 nm, orange light has a wavelength of about 590 nm, red light has a wavelength of about 650 nm. Other types of labels that generate signals in the non-visible spectrum of the electromagnetic spectrum also can be used and include, for example, signals within wavelengths of the ultraviolet region between about 50-350 nm, other areas of the visible portion between about 350-800 nm, the near-infrared region between about 700-2500 nm, the infrared region between about 800-3000 nm as well as longer and shorter wavelengths.

Particularly useful fluorescent labels having emissions across the visible spectrum include, for example, Alexa fluor Dyes commercially available from Invitrogen (see, for example, the URL probes.invitrogen.com/handbook/tables/0329.html). Labels within this exemplary family include, for example, Alexa350 which emits blue light at 442 nm, Alexa 405 emitting blue light at 421 nm, Alexa430 emitting yellow-green light at 539 nm, Alex488 emitting green light at 519 nm, Alexa500 emitting green light at 525 nm, Alexa 514 emitting yellow-green light at 540 nm, Alexa532 emitting yellow light at 554 nm, Alex546 emitting orange light at 573 nm, Alexa555 emitting red-orange light at 565 nm, Alexa 568 emitting red-orange light at 603 nm, Alexa594 emitting red light at 617 nm, Alexa610 emitting red light at 628 nm, Alexa633 emitting far-red at 647 nm, Alexa635 emitting far-red at 647 nm, Alexa647 emitting far-red light at 668 nm, Alexa680 emitting near-infrared light at 690 nm, Alexa700 emitting near-infrared light at 723 nm and Alexa750 emitting near-infrared light at 775 mm.

In a preferred embodiment, a secondary label is used. A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, etc. Purification tags or affinity labels are examples of secondary labels. Secondary labels find particular use in systems requiring separation of labeled and unlabeled probes. Secondary labels include, but are not limited to, one of a binding partner pair; chemically modifiable moieties; nuclease inhibitors, enzymes such as horseradish peroxidase, alkaline phosphatases, luciferases, etc. In a preferred embodiment, the secondary label is a member of a binding partner pair. For example, the label may be a hapten or antigen, which will bind its binding partner. Suitable binding partner pairs include, but are not limited to those set forth above in regard to purification tags or affinity labels.

Non-limiting examples of label moieties useful for detection in the methods of the invention include, without limitation, suitable enzymes such as horseradish peroxidase, alkaline phosphatase, β-galactosidase and/or acetylcholinesterase; members of a binding pair that are capable of forming complexes such as streptavidin/biotin, avidin/biotin and/or an antigen/antibody complex including, for example, rabbit IgG and anti-rabbit IgG; fluorophores such as umbelliferone, fluorescein, fluorescein isothiocyanate, rhodamine tetramethyl rhodamine, eosin, green fluorescent protein, erythrosin, coumarin, methyl coumarin, pyrene, malachite green, stilbene, lucifer yellow, Cascade Blue™, Texas Red, dichlorotriazinylamine fluorescein, dansyl chloride, phycoerythrin, fluorescent lanthanide complexes such as those including Europium and Terbium, Cy3, Cy5, molecular beacons and fluorescent derivatives thereof, as well as others known in the art as described, for example, in Principles of Fluorescence Spectroscopy, Joseph R. Lakowicz (Editor), Plenum Pub Corp, 2nd edition (July 1999) and the 6th Edition of the Molecular Probes Handbook by Richard P. Hoagland; a luminescent material such as luminol; light scattering or plasmon resonant materials such as gold or silver particles or quantum dots; or radioactive material include ¹⁴C, ¹²³I, ¹²⁴I, ¹²⁵I, ¹³¹I, Tc⁹⁹m, ³⁵S or ³H.

In a preferred embodiment, the secondary label is a chemically modifiable moiety. In this embodiment, labels comprising reactive functional groups are incorporated into the nucleic acid. The functional group can then be subsequently labeled with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference).

Labeling can include a signal amplification technique. Signal amplification can be carried out, for example, using streptavidin-phycoerythrin (SAPE) and a biotinylated anti-SAPE antibody. In one embodiment, a three step protocol can be employed in which nucleic acids that have been modified to incorporate biotin are first incubated with streptavidin-phycoerythrin (SAPE), followed by incubation with a biotinylated anti-streptavidin antibody, and finally incubation with SAPE again. This process creates a cascading amplification sandwich since streptavidin has multiple antibody binding sites and the antibody has multiple biotins. Those skilled in the art will recognize from the teaching herein that other receptors such as avidin, modified versions of avidin, or antibodies can be used in an amplification complex and that different labels can be used such as Cy3, Cy5 or others set forth previously herein. Another example of signal amplification uses nucleic acids labeled with a dinitrophenyl (DNP) moiety that can be detected by an antibody that is labeled with a fluorophore. Further exemplary signal amplification techniques and components that can be used in the invention are described, for example, in U.S. Pat. No. 6,203,989 B1. Biotin or DNP can be introduced into a nucleic acid using biotin labeled nucleotides or DNP labeled nucleotides, respectively, such as those commercially available from PerkinElmer or Roche.

In particular embodiments, the identity of a target small nucleotide sequence is determined by detecting the molecular weights of the amplification product or a fragment thereof, such as by chromatography or mass spectroscopy.

Mass spectrometry techniques for use in the present invention include collision-induced dissociation (CID) fragmentation analysis (e.g., CID in conjunction with a MS/MS configuration, see Schram, K. (1990) Biomedical Applications of Mass Spectrometry 34:203-287; and Crain P. (1990) Mass Spectrometry Reviews 9:505-554); fast atomic bombardment (FAB mass spectrometry) and plasma desorption (PD mass spectrometry), see Koster et al. (1987) Biomedical Environmental Mass Spectrometry 14:111-116; and electrospray/ionspray (ES) and matrix-assisted laser desorption/ionization (MALDI) mass spectrometry (see Fenn et al. (1984) J. Phys. Chem. 88:4451-4459, Smith et al. (1990) Anal. Chem. 62:882-889, and Ardrey, B. (1992) Spectroscopy Europe 4:10-18). MALDI mass spectrometry is particularly well suited to such analyses when a time-of-flight (TOF) configuration is used as a mass analyzer (MALDI-TOF). See International Publication No. WO 97/33000, published Sep. 12, 1997, see also Huth-Fehre et al. (1992) Rapid Communications in Mass Spectrometry 6:209-213, and Williams et al. (1990) Rapid Communications in Mass Spectrometry 4:348-351.

In this regard, a number of mass tags suitable for use with nucleic acids are known (see U.S. Pat. No. 5,003,059 to Brennan and U.S. Pat. No. 5,547,835 to Koster), including mass tags which are cleavable from the nucleic acid (see International Publication No. WO 97/27331).

In certain instances, a plurality of nucleic acids can be deconvoluted by chromatographic techniques prior to detection by mass spectroscopy. For example, prior to introducing a sample into the spectrometer, the mixture can first be at least semi-purified. Separation procedures based on size (e.g. gel-filtration), solubility (e.g. isoelectric precipitation) or electric charge (e.g. electrophoresis, isoelectric focusing, ion exchange chromatography) may be used to separate a mixture of amplimers. A preferred separation procedure is high performance liquid chromatography (HPLC). These same separation procedures can be used on their own or in various combinations without mass spectroscopy to determine the molecular weight of a detected amplification product or associated label in a method of the invention.

In another embodiment, the hybridization tags are detected on micro-formatted multiplex or matrix devices (e.g., DNA chips) (see M. Barinaga, 253 Science, pp. 1489, 1991; W. Bains, 10 Bio/Technology, pp. 757-758, 1992). These methods usually attach specific DNA sequences to very small specific areas of a solid support, such as micro-wells of a DNA chip. Typically, an oligonucleotide is linked to a solid support and a tag nucleic acid is hybridized to the oligonucleotide. Either the oligonucleotide, or the tag, or both, can be labeled, typically with a fluorophore. Where the tag is labeled, hybridization is detected by detecting bound fluorescence. Where the oligonucleotide is labeled, hybridization is typically detected by quenching of the label. Where both the oligonucleotide and the tag are labeled, detection of hybridization is typically performed by monitoring a color shift resulting from proximity of the two bound labels. A variety of labeling strategies, labels, and the like, particularly for fluorescent based applications are described, supra.

The present invention provides methods for detecting the presence or absence of small target nucleic acid sequences in a sample. In particular embodiments, the amount of one or more small target nucleotide sequence that is present in a sample can be determined either as an absolute amount or relative amount compared to one or more other nucleotide sequence. A nucleic acid sequence of the present invention will generally contain phosphodiester bonds, although in some cases, as outlined below (for example in the generation of the probes of the invention), nucleic acid analogs are included that may have alternate backbones, comprising, for example, those described in US 2005/0181394, which is incorporated herein by reference.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides as well as nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

As outlined herein, in particular embodiments the target sequence can include a position for which sequence information is desired, generally referred to herein as the “detection position” or “detection locus”. In a preferred embodiment, the detection position is a single nucleotide, although in some embodiments, it may comprise a plurality of nucleotides, either contiguous with each other or separated by one or more nucleotides. By “plurality” as used herein is meant at least two. As used herein, the base which basepairs with a detection position base in a hybrid is termed a “readout position” or an “interrogation position”; thus target-specific probes of the invention can comprise an interrogation position.

In some embodiments, as is outlined herein, the target sequence may not be the sample target sequence but instead is a product of a reaction herein, sometimes referred to herein as a “secondary” or “derivative” target sequence, or an “amplicon”. Examples of such reaction products include, but are not limited to, a cDNA such as those produced using methods described herein with regard to FIGS. 1 and 2; an extension product of a target-specific probe such as those produced using methods described herein with regard to FIGS. 3 and 4 or an amplicon produced from an extension product of a target-specific probe such as those produced using methods described herein with regard to FIGS. 3 and 4.

In particular embodiments, a single target nucleic acid sequence is detected. If desired, a plurality of sequences can be detected, for example, in a multiplex format. “Multiplexing” refers to the detection, analysis or amplification of a plurality of targets in a single sample, typically simultaneously. The present invention is useful for detection of a single target sequence as well as a plurality of target sequences. In addition, as described below, the methods of the invention can be performed simultaneously and in parallel in a large number of samples. As used herein, “plurality” or grammatical equivalents herein refers to at least 2, 50, 100, 200, 500, 1000, 5000, 10,000, 50,000 100,000 or 1,000,000 different target sequences. Detection is performed on any of a variety of platforms as described herein or otherwise known in the art.

In one embodiment the invention is directed to a method for determining the expression level of a small target nucleotide sequence in a sample by contacting nucleic acid molecules derived from a sample with a set of probes under conditions where perfectly complementary probes form a hybridization complex with the target sequence, each of the probes comprising at least one universal priming site and a target-specific sequence; amplifying the probes forming the hybridization complexes to produce amplicons; and detecting the amplicons, wherein the detection of the amplicons indicates the presence of the target sequence in the sample; and determining the expression level of the target sequence.

In a preferred embodiment the non-hybridized nucleic acids are removed by washing. In this embodiment the hybridization complexes can be immobilized on a solid support and washed under conditions sufficient to remove non-hybridized nucleic acids, i.e. non-hybridized probes and sample nucleic acids. In a particularly preferred embodiment immobilized complexes are washed under conditions sufficient to remove imperfectly hybridized complexes. That is, hybridization complexes that contain mismatches are also removed in the wash steps.

A variety of hybridization or washing conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10 C lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of helix destabilizing agents such as formamide. The hybridization or washing conditions may also vary when a non-ionic backbone, i.e. PNA is used, as is known in the art. In addition, cross-linking agents may be added after target binding to cross-link, i.e. covalently attach, the two strands of the hybridization complex.

In one embodiment the hybridization complexes are immobilized by binding of a purification tag to the solid support. That is, a purification tag is incorporated into the nucleic acids. Purification tags can be incorporated into nucleic acids in a variety of ways. In one embodiment probes or primers contain purification tags as described herein. That is, the probe is synthesized with a purification tag, i.e. biotinylated nucleotides, or a purification tag is added to the probe. Thus, upon hybridization with target nucleic acids, immobilization of the hybridization complexes is accomplished by a purification tag. The purification tag associates with the solid support. Similar configurations and synthetic methods can be used to incorporate other types of labels into a nucleic acid.

The purification tag also can be incorporated into a nucleic acid following a primer extension reaction. Briefly, following hybridization of one or more primers with target nucleic acids, a polymerase extension reaction is performed. In this embodiment tagged nucleotides, such as biotinylated nucleotides, are incorporated into the primer extension product as a result of the polymerase catalyzed reaction. That is, once the target sequence and the first probe sequence have hybridized, the method of this embodiment further comprises the addition of a polymerase and at least one nucleotide (dNTP) labeled with a purification tag.

In addition, the purification tag can be incorporated into the target nucleic acid. In this embodiment, the target nucleic acid is labeled with a purification tag and immobilized to the solid support as described above. Preferably the tag is biotin. Once formed, the tagged extension product is immobilized on the solid support as described above. Once immobilized, the complexes are washed so as to remove unhybridized nucleic acids.

In another embodiment, the methods of the invention for detecting a target sequence can include, for example, the step of generating a report on the results of the target sequence or target sequences detected. For example, the report can indicate whether a target sequence was present or absent, the relative amount of a target sequence or its quantitative amount as well as all other characteristics or attributes of the target sequence, the conditions employed, the target-specific probes employed, the configuration of the method, the format of the assay as well as various other permutations or considerations evaluated or not evaluated. A target sequence can be identified in a report, for example, by its presence or absence in a sampled assayed, by sequence, location on a chromosome or by a name of a locus. Alternatively, the report can include data obtained from a method of the invention in a format that can be processed or analyzed to identify one or more detected target sequences. The methods of the invention can further provide a report that includes, for example, a correlation or predictive outcome of a detected target sequence to a disease or species characteristic. Similarly, such reports and preparation of such reports can be included in any of the methods of the invention.

Thus, the invention further provides a report of at least one result obtained by a method of the invention. A report of the invention can be in any of a variety of recognizable formats including, for example, an electronic transmission, computer readable memory, an output to a computer graphical user interface, compact disk, magnetic disk or paper. Other formats suitable for communication between humans, machines or both can be used for a report of the invention. The report, whether in preliminary, intermediate or final form, can be analyzed by human or machine or both for use or dissemination of the target sequence information contained therein. Therefore, a further embodiment of the invention is the step of evaluating a report generated on the detection results of a target sequence or target sequences.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

The entire contents of all of the references (including literature references, issued patents, published patent applications, and co-pending patent applications) cited throughout this application are expressly incorporated by reference herein.

### EXAMPLE I

### Multiplex MicroRNA Amplification

This Example demonstrates methods for modifying small RNA molecules to include universal priming sites, amplification of the modified small RNA molecules and detection of the resulting amplicons.

Total RNA samples obtained from PC3, MCF7, 293 or Hela cells were purchased from Ambion (Austin, Tex.). The samples were subjected to the two methods set forth below.

Two methods were used to attach a universal oligo sequence to 3′ end of small RNA species present in the RNA samples. In the first method, a 5′ phosphorylated chimera oligo was ligated to the 3′ end of RNA using T4 RNA ligase according to the manufacturer's instructions (Promega, Madison, Wis.; Cat # M1051). The 5′ end of the chimera contained 5 RNA bases, followed by 16 DNA bases of a first universal priming site at the 3′ end. In addition, the 3′ end of the oligo was modified with an inverted 3′-3′ bond to prevent self ligation. A biotin labeled primer, having a sequence complementary to the first universal priming site was then added to the modified RNA sample along with thermo-stable RT enzyme and cDNA synthesis was carried out. The cDNA synthesis was carried out at high temperature (60° C.) to open up potential secondary structures existing in miRNAs (in separate experiments, other RT enzymes performed well at 42° C. as well.). A diagrammatic representation of this first approach is shown in FIG. 1.

Thus, the invention provides a method for determining the presence of a small target ribonucleotide sequence in a sample, wherein the method includes (a) modifying ribonucleotide species in the sample by adding a chimera nucleic acid to the 3′ end of the ribonucleotide species, wherein the chimera nucleic acid comprises RNA bases at its 5′ end and DNA bases at its 3′ end; (b) converting the modified ribonucleotide species into a plurality of complementary DNA (cDNA) sequences; (c) immobilizing the plurality of cDNA sequences to a solid support; (d) contacting the immobilized cDNA species with a pool of probe nucleic acids under conditions that allow sequence specific annealing, wherein each probe nucleic acid corresponds to a small target ribonucleotide sequence; (e) extending the probe nucleic acids in a manner complementary to the immobilized cDNA species; (f) removing the extended probe nucleic acids from the immobilized cDNA species; (g) amplifying the extended probe nucleic acids to generate amplicons, and (h) detecting the amplicons, wherein detection of each amplicon indicates the presence of a small target ribonucleotide sequence.

In the second method, the RNA sample was polyadenylated using Poly(A) Polymerase I (PAP) enzyme (Ambion, cat # AM2030). In this way, a poly A sequence was added to the 3′ end of the RNA molecules. The length of the poly A-stretch was controlled by varying the amount of PAP enzyme to achieve a poly-A tail in an average range of 20 nucleotides. A biotin labeled primer, having a poly T sequence at the 3′ end and the first universal priming site at the 5′ end, was then added to the modified RNA sample along with thermo-stable RT enzyme and cDNA synthesis was carried out as described above for the first approach. A diagrammatic representation of this second approach is shown in FIG. 2.

The cDNA samples derived from either method described above (referred to as the “first strand” cDNA below) was subjected to solid phase second strand extension as shown in FIG. 3 and set forth below. A mixture of miRNA-specific assay oligos was annealed gradually to the first strand cDNA template in the presence of streptavidin beads. Under these conditions the biotinylated first strand cDNA is immobilized on the beads. Each miRNA-specific assay oligo in the mixture had three separate portions including in order from 3′ to 5′ (1) a sequence of 18-22 nucleotides that was complementary to a known miRNA sequence, (2) an address sequence of 22 nucleotides, and (3) a second universal priming site of 18 nucleotides. DNA polymerase was added for a 15 second reaction at 45° C. to extend the annealed miRNA-specific assay oligos. The resulting second strand cDNA included from 3′ to 5′ (1) the first universal priming site, (2) the poly T sequence or 5 nucleotides from the RNA sequence of the chimeric oligo; (3) the target miRNA sequence, (4) the address sequence and (5) the second universal priming site.

The immobilized double stranded cDNA washed to remove unbound oligos and other reaction components. The second strand was eluted from the immobilized first strand by high temperature denaturation and used as a template for PCR using first and second universal primers that annealed to the universal priming sites flanking the target miRNA sequence and address sequence as shown in FIG. 3. One of the universal primers was labeled with Cy3 dye and the other was labeled with biotin.

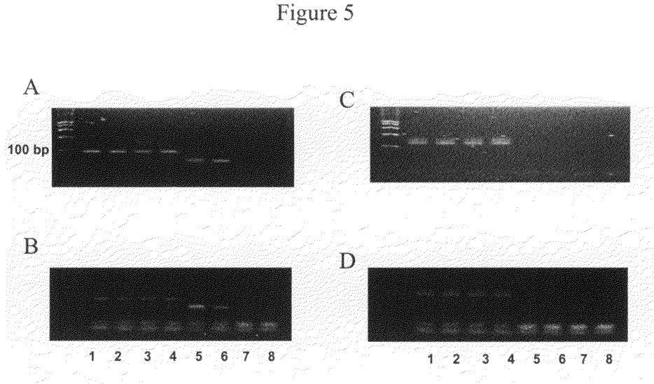

Products of the above reactions were separated by agarose gel electrophoresis. Results are shown in FIG. 5. A gel loaded with products of the first method, in which first strand cDNA was obtained by oligo ligation, is shown in panel A (ethidium bromide staining and detection) and panel B (Cy3 detection). A gel loaded with products of the second method, in which first strand cDNA was obtained by polyadenylation, is shown in panel C (ethidium bromide staining and detection) and panel D (Cy3 detection). For each gel, lanes 1, 2, 3, and 4 correspond to results obtain with PC3, MCF7, 293 and Hela RNAs, respectively. Lanes 5, 6, 7, and 8 correspond to various negative controls including polyadenylation control (or chimeric oligo control), RT-control, assay oligo annealing control and PCR control, respectively.

As shown in FIG. 5, PCR products with the correct size (˜100 bp) and the right dye labeling (Cy3, Green color) were present in lanes 1-4, but not lanes 5-8 indicating that specific modification and amplification was achieved.

### EXAMPLE II

### MicroRNA Expression Profiling Using Universal Bead Arrays

This example demonstrates sensitive and reproducible expression profiling of microRNA species.

Dye labeled amplification products were obtained using the polyadenylation-based amplification method described in Example I. Briefly, a solid-phase primer extension step was carried out after assay oligos were annealed to immobilized cDNAs in order to enhance the discrimination among homologous miRNA sequences. In addition, universal PCR was used to amplify all targets prior to array hybridization. The solid-phase cDNA selection and enzymatic 3′-end mismatch discrimination in the primer extension step enhance the discrimination among homologous miRNA sequences and provide the assay with high specificity. The universal PCR amplification provides the assay with high sensitivity. PCR primers are shared among all target sequences, and amplicons are a uniform size. This allows unbiased amplification of the ligated oligo population.

The dye labeled amplification products were hybridized to a universal array, and fluorescence intensity is measured for each bead. The universal array was a Sentrix® Array Matrix available from Illumina (San Diego, Calif.). The arrays have 1,624 different elements at an average redundancy of 30 beads of each type. The fiber bundles are arranged in the geometry of a 96-well microtiter plate to produce a Sentrix® Array Matrix capable of 96×1536=147,456 assay data points. Hybridization and detection was carried out according to the manufacturer's instructions for the DASL assay (Illumina, San Diego, Calif.).

The assays were designed to simultaneously analyze either 470 well-annotated human miRNAs or 380 mouse miRNAs (miRBase: microrna.sanger.ac.uk/), and additional 273 human miRNAs compiled from the scientific literature. One specific assay probe was designed against each mature miRNA sequence, each having a unique address sequence. Thus, a given address sequence was uniquely associated with a miRNA sequence. Each unique address sequence was complementary to a capture sequence immobilized on the universal array. All of the human or mouse miRNAs were assayed simultaneously.

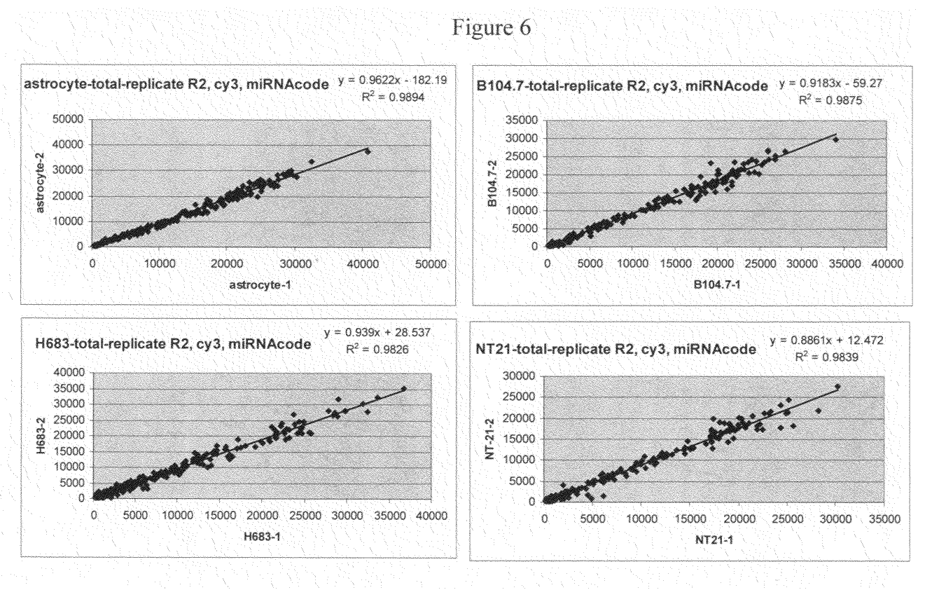

Assays were run using 200 ng of input RNA from four different cell types. For each sample two technical replicates were run (i.e. the two samples were independently processed starting from target modification to amplification and detection). As shown in FIG. 6, highly reproducible expression profiles (R²>0.98) were obtained between technical replicates, using 200 ng total RNA input. These results show that it possible to profile miRNA expression in cancer tissue samples. Furthermore, very similar expression profiles were obtained between total RNA and enriched small RNA species (R²=0.97).

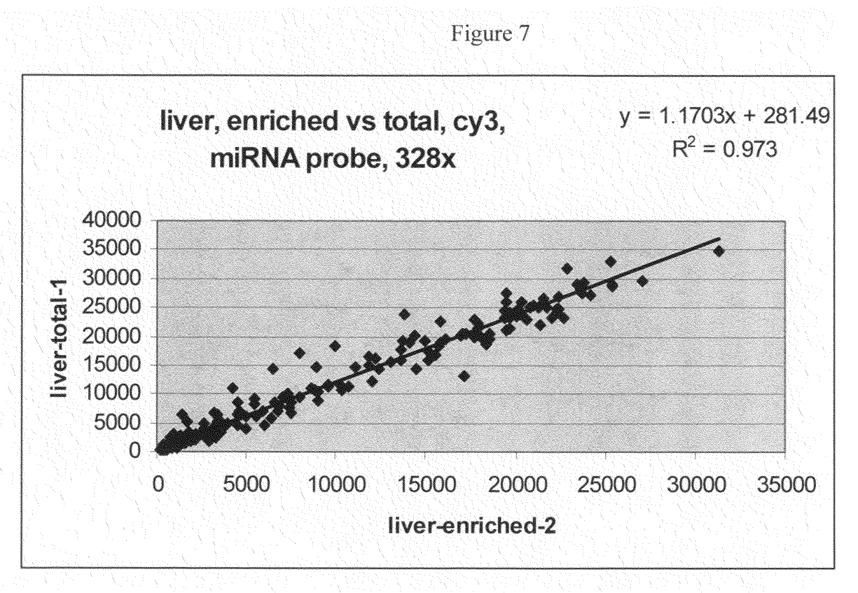

Assays were run using as input either 200 ng of total RNA from liver tissue or enriched RNA obtained from 1 μg of total RNA from liver cells. The Invitrogen PureLink miRNA isolation kit (cat# K1570) was used to enrich the small RNAs. As shown in FIG. 7, highly comparable expression profiles (R²>0.97) are obtained between total RNA (200 ng) and enriched small RNA (equal to 1 μg of total RNA) inputs.

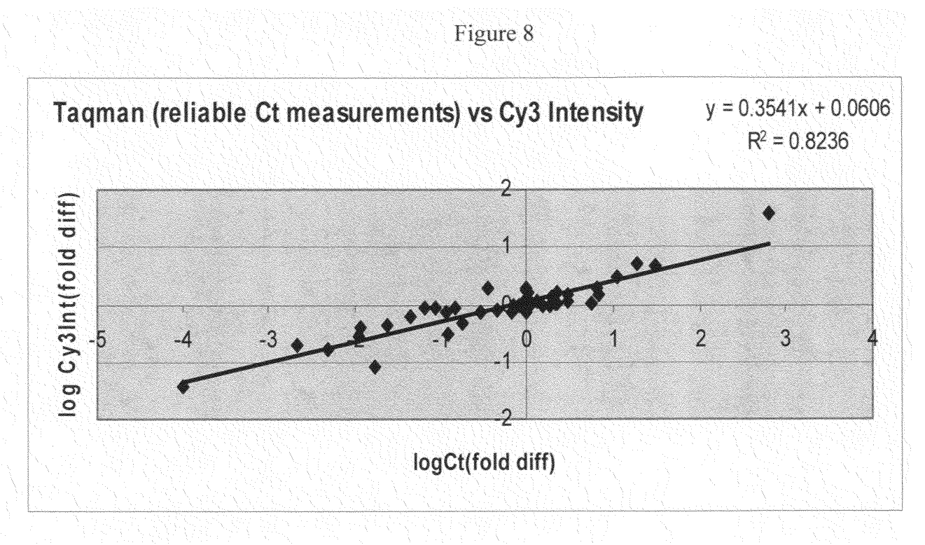

For comparison with another method, expression levels of 33 miRNAs were measured in four different cancer cell lines (PC3, 293, MCF7 and Hela) by a stem-loop based RT-PCR method. As shown in FIG. 8, high concordance (R²=0.82) was obtained between results obtained using an Illumina miRNA array and results using RT-PCR. The logarithmic fold difference in abundance in pairwise comparisons between four cancer cell lines (PC3, 293, MCF7 and Hela) was estimated for 33 miRNAs in both the Illumina assay (fold difference in array intensity, y-axis) and RT-PCR (fold difference in abundance derived from crossover threshold, x-axis). Thus, high concordance was obtained between the array results and the RT-PCR results, when “fold-difference” was compared.

The results described above demonstrate that the methods for microRNA expression profiling using universal bead arrays is useful for high-throughput expression profiling of miRNA in large numbers of cell line or tissue samples.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A method for determining the presence of a small target ribonucleotide sequence in a sample, said method comprising
(a) modifying ribonucleotide species contained in the sample by polyadenylating the 3′ ends;
(b) converting the polyadenylated ribonucleotide species into a plurality of complementary DNA (cDNA) sequences;
(c) immobilizing said plurality of cDNA sequences to a solid support;
(d) contacting said immobilized cDNA species with a pool of probe nucleic acids under conditions that allow sequence specific annealing, wherein each probe nucleic acid corresponds to a small target ribonucleotide sequence;
(e) extending said probe nucleic acids in a manner complementary to the immobilized cDNA species;
(f) removing the extended probe nucleic acids from the immobilized cDNA species;
(g) amplifying the extended probe nucleic acids to generate amplicons, and
(h) detecting said amplicons, wherein detection of each amplicon indicates the presence of a small target ribonucleotide sequence.

2. The method of claim 1, further comprising an initial step of enriching said sample for small RNA species.

3. The method of claim 2, wherein said small RNA species comprise ncRNA.

4. The method of claim 1, wherein said sample comprises purified RNA.

5. The method of claim 2, wherein said small target ribonucleotide sequences comprise micro RNA (miRNA).

6. The method of claim 1, wherein said miRNA sequences are less than 30 nucleotides.

7. The method of claim 1, wherein said polyadenylation step adds at least 15 nucleotides.

8. The method of claim 1, wherein the sample comprises purified small RNA species.

9. The method of claim 1, wherein said cDNA sequence is obtained using a labeled primer comprising a sequence complementary to the 3′ end of the polyadenylated ribonucleotide species and further comprising a label.

10. The method of claim 9, wherein said label has affinity for said solid support.

11. The method of claim 10, wherein said label comprises biotin.

12. The method of claim 1, wherein each probe nucleic acid comprises a unique address sequence and a universal primer sequence.

13. The method of claim 1, wherein said unique address sequence is complementary to a capture sequence on an array.

14. The method of claim 13, wherein said detection of the amplicons comprises capture on the array.

15. The method of claim 14, wherein said capture comprises binding of said unique address sequences to said capture sequences.

16. A method for determining the presence of a small target ribonucleotide sequence in a sample, said method comprising
(a) modifying ribonucleotide species in the sample by adding a chimera nucleic acid to the 3′ end of the ribonucleotide species, wherein said chimera nucleic acid comprises RNA bases at its 5′ end and DNA bases at its 3′ end;
(b) converting the modified ribonucleotide species into a plurality of complementary DNA (cDNA) sequences;
(c) immobilizing said plurality of cDNA sequences to a solid support;
(d) contacting said immobilized cDNA species with a pool of probe nucleic acids under conditions that allow sequence specific annealing, wherein each probe nucleic acid corresponds to a small target ribonucleotide sequence;
(e) extending said probe nucleic acids in a manner complementary to the immobilized cDNA species;
(f) removing the extended probe nucleic acids from the immobilized cDNA species;
(g) amplifying the extended probe nucleic acids to generate amplicons, and
(h) detecting said amplicons, wherein detection of each amplicon indicates the presence of a small target ribonucleotide sequence
